(19) United States
(12) Patent Application Publication
(10) Pub. No.: US 2016/0235446 A1
(43) Pub. Date: Aug. 18, 2016

(54) Spinal Rods and Methods

(71) Applicant: SpineCraft, LLC, Westmont, IL (US)

(72) Inventor: Wagdy W. Asaad, Burr Ridge, IL (US)

(73) Assignee: SpineCraft, LLC, Westmont, IL (US)

(21) Appl. No.: 14/989,010

(22) Filed: Jan. 6, 2016

Related U.S. Application Data
(60) Provisional application No. 62/116,482, filed on Feb. 15, 2015.

(51) Int. Cl. A61B 17/70

(52) U.S. Cl. CPC A61B 17/7004

(57) ABSTRACT

A spinal rod is provided that includes an elongate body having first and second ends, a length and an outside diameter. The outside diameter is substantially the same size over an entirety of the length of the elongate body. A cavity extends within the elongate body over a cavity length. The cavity length is less than the overall length of the elongate body. A stiffness of the elongate body in portions containing the cavity is less than a stiffness of a portion of the elongate body not containing the cavity.

### CROSS-REFERENCE

This application is a claims the benefit of U.S. Provisional Application No. 62/116,482, filed on Feb. 15, 2015, which application is incorporated herein, in its entirety, by reference thereto.

### FIELD OF THE INVENTION

The present invention relates to the field of spinal surgery. More particularly, the present invention relates to spinal rods used in spinal surgery and methods of using the spinal rods.

### BACKGROUND OF THE INVENTION

Spinal rods are commonly used in the surgical treatment of spinal disorders such as degenerative disc disease, scoliosis, fractures, other curvature abnormalities of the spine, other degenerative diseases of the spine, other diseases of the spine and other traumatic injuries to the spine. In performing fusion of two or more levels of the spine, and particularly in long fusion procedures where more than two levels are fused, it has been reported that accelerated degeneration of segments (levels) adjacent to those levels being fused has occurred, e.g., see Chou et al., “Adjacent segment degeneration after lumbar spinal posterolateral fusion with instrumentation in elderly patients”, Arch Orthop Trauma Surg (2002) 122:39-43; Hilibrand et al., “Adjacent segment degeneration and adjacent segment disease: the consequences of spinal fusion?”, The Spine Journal, Vol. 4, Issue 6, Supplement, pp S190-S194, Nov-Dec 2004; and Kumar et al., “Long-term follow-up of functional outcomes and radiographic changes at adjacent levels following lumbar spine fusion for degenerative disc disease”, Eur Spin J (2001) 10:309-313, each of which is hereby incorporated herein, in its entirety, by reference thereto.

Accelerated segment degeneration is often noted particularly in the level or levels immediately above the fusion. For example, in a long fusion procedure fusing levels from the lower lumbar region to the upper thoracic region, the one or more levels immediately above the fusion (e.g., upper thoracic or cervical vertebra(ae)) have been found to rapidly degenerate, sometimes within a matter of weeks after the fusion procedure has been performed.

A major contributing factor to this accelerated degeneration is believed to be the abrupt transition from a very high stiffness provided at the top end of the spinal rod(s) implanted to rigidify the levels to be fused and the much lower stiffness of the level(s) adjacent the end of the rod(s) which are not fused. Attempts have been made at reducing the stress transition, each with its own drawbacks.

Simonson in U.S. Patent Application Publication No. 2012/0290013 discloses a tapered spinal rod that varies in cross-sectional diameter from 6.5 mm at the bottom to 3 mm at the top. A problem with this configuration is that, at almost every level of fusion, the screws used to fix the rods to the vertebrae do not set flat against the rods because of the taper of the external surfaces of the rods. This results in the unfortunate disadvantage that not every level of the fusion can be effectively locked to the rods.

Patterson et al. in U.S. Pat. No. 7,875,059 discloses spinal support members having various stiffnesses in which different portions of the support member are formed of different materials, with the different material having different stiffness characteristics. Problems with these designs include the greater cost of manufacture, compared to a support member made uniformly of one material, and, more importantly, and are more difficult to manufacture as the different components must be joined, by bonding or molding. More important, these designs run the risk of delamination after implantation in a patient, which can cause injury and/or require additional surgery for replacement or repair.

Other approaches have attempted to provide a dynamic fixation by one or more rods, at one or two levels above the fusion by providing a rod that bends or polymer rod (or rod extension, such as in Patterson) that can bend or flex. This requires the use of pedicle screws in the levels above the fusion that would otherwise not be required. As these levels above the fusion are smaller vertebrae, the patient can sometimes feel the pedicle screws protruding against the skin, causing discomfort. In some cases, the pedicle screws are even visible through the skin as they protrude into the layers of the skin.

There is a continuing need for improved products designed to reduce or eliminate the accelerated degeneration of segments adjacent to fused segments of a spine.

### SUMMARY OF THE INVENTION

According to an aspect of the present invention, a spinal rod having an elongate body having first and second ends, a length and an outside diameter is provided. The outside diameter is substantially the same size over an entirety of the length of the elongate body. A cavity extends within the elongate body over a cavity length; wherein the cavity length is less than the length of the elongate body; and wherein a stiffness of the elongate body in portions containing the cavity is less than a stiffness of a portion of the elongate body not containing the cavity.

In at least one embodiment, the cavity length is in the range of 10% to 50% of the length of the elongate body.

In at least one embodiment, the cavity length is in the range about 15% to about 35% of the length of the elongate body.

In at least one embodiment, the cavity length is less than 50% of the length of the elongate body.

In at least one embodiment, the cavity extends from the first end to a location intermediate of the first and second ends.

In at least one embodiment, a diameter of the cavity varies along a length of the cavity.

In at least one embodiment, the cavity includes stepped portions, each stepped portion having a diameter that is substantially constant over a length of the respective step portion, and wherein the diameters of the stepped portions are each different from one another.

In at least one embodiment, the diameter of a first of the stepped portions is larger than the diameters of all other of the stepped portions and extends from the first end of the main body.

In at least one embodiment, the diameters of the stepped portions become decreasingly smaller in a direction from the first of the stepped portions toward the second end of the elongate body.

In at least one embodiment, the diameter of the cavity varies continuously over a length thereof.

In at least one embodiment, the diameter of the cavity decreases from a location nearest the first end of the elongate body to a location nearest the second end of the elongate body.

In at least one embodiment, the spinal rod is made entirely of a single material.

In at least one embodiment, the single material is metal.

In at least one embodiment, the metal comprises titanium.

In at least one embodiment, the metal comprises Cobalt Chromium.

In at least one embodiment, the single material comprises polymer.

In at least one embodiment, a first portion of the elongate body contains the cavity and a second portion of the elongate body is solid, and wherein the first portion has a stiffness in a range of from about 10 N/mm to about 250 N/mm and the second portion has a stiffness in a range of from about 40 N/mm to about 1000 N/mm.

In at least one embodiment, a first portion of the elongate body contains the cavity and a second portion of the elongate body is solid. The second portion has a stiffness in a range of from about 40 N/mm to about 1000 N/mm The first portion has at least first and second sub-portions, such that the first sub-portion portion has a stiffness in a range of from about 20 N/mm to about 500 N/mm and the second sub-portion has a stiffness in a range of from about 10 N/mm to about 250 N/mm, wherein the stiffness of the first sub-portion is greater than the stiffness of the second sub-portion.

In at least one embodiment, the first-sub portion is located intermediate of the second portion and the second sub-portion.

In at least one embodiment, a first portion of the elongate body contains the cavity and a second portion of the elongate body is solid, and wherein the second portion has a stiffness in a range of from about 14 GPa to about 250 GPa and wherein the first portion has at least first and second sub-portions, such that the first portion has a stiffness that varies from a range of from about 7 GPa to about 125 GPa in one of the at least first and second sub-portions to a range of from about 8.75 GPa to about 155 GPa in another of the at least first and second sub-portions.

In at least one embodiment, the spinal rod further includes a marker on an external surface of the elongate body, the marker identifying a location intermediate the first and second ends where the cavity ends.

In another aspect of the present invention, a method of performing spinal fusion is provided, including: selecting a spinal rod comprising an elongate body having first and second ends, a length and an outside diameter, the outside diameter being substantially the same size over an entirety of the length of the elongate body; and a cavity extending within the elongate body over a cavity length, wherein the cavity length is less than the length of the elongate body; and fixing the spinal rod to at least two adjacent spinal vertebrae.

In at least one embodiment, the fixing comprises fixing the spinal rod to three or more adjacent vertebrae.

In at least one embodiment, the fixing comprises orienting the spinal rod so that a portion of the elongate body containing the cavity is above a portion of the elongate body that does not contain the cavity.

In at least one embodiment, the fixing comprises fixing the spinal rod to each of the vertebrae spanned by the elongate body.

These and other features of the invention will become apparent to those persons skilled in the art upon reading the details of the embodiments as more fully described below.

### BRIEF DESCRIPTION OF THE DRAWINGS

FIG. 1A illustrates a spinal rod according to an embodiment of the present invention.

FIG. 1B is a view of FIG. 1A in which an upper portion of the elongate body is shown as a longitudinal sectional view.

FIG. 1C is a cross-sectional view of FIG. 1A taken along line 1C-1C.

FIG. 1D is a cross-sectional view of FIG. 1A showing the solid lower portion of the elongate body.

FIG. 2 illustrates a spinal rod according to another embodiment of the present invention.

FIG. 3 illustrates a spinal rod according to another embodiment of the present invention.

FIGS. 4A and 4B are partial views showing variants of the embodiment of FIG. 3.

FIG. 5 illustrates a pair of spinal rods having been implanted on a spine of a patient according to an embodiment of the present invention.

FIG. 6 illustrates a spinal rod that is curved along its length, according to an embodiment of the present invention.

### DETAILED DESCRIPTION OF THE INVENTION

Before the present devices and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a vertebra” includes a plurality of such vertebrae and reference to “the rod” includes reference to one or more rods and equivalents thereof known to those skilled in the art, and so forth.

The publications discussed herein are provided solely for their disclosure prior to the filing date of the present application. The dates of publication provided may be different from the actual publication dates which may need to be independently confirmed.

A “level” as used herein refers to fusion of two adjacent vertebrae to stop the motion at one segment, i.e. intervertebral disc; for example, an L4-L5 fusion is a one-level spinal fusion.

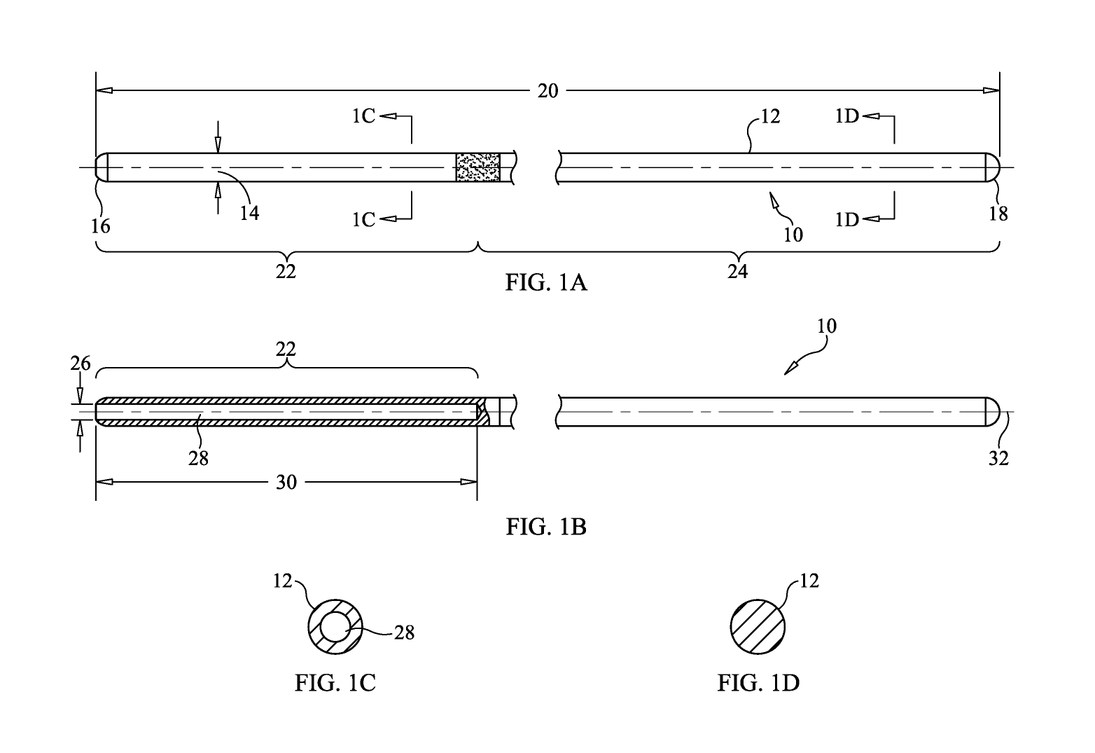

FIG. 1A is a plan view of a spinal rod 10 according to an embodiment of the present invention. Spinal rod 10 comprises an elongate body 12 that is preferably formed as an integral unit so as to avoid risk of disintegration of parts during use of the rod 10 after implantation within a patient. Rod 10, as well as all other embodiments described herein is preferably formed of metal, but could alternatively be formed of polymer or polymer composite. Preferably rod 10 and all other embodiments are formed of titanium or titanium alloy, but could alternatively be made from cobalt-chromium alloy, stainless steel, nickel-titanium, polymer such as polyetheretherketone (PEEK), carbon-reinforced PEEK or other metal, metal alloy, polymer or polymer alloy that is biocompatible and provides the required strength and other material properties required to function for this purpose.

Elongate body 12 is manufactured to have the same outside diameter 14 over the entire length thereof, except for the radiused ends 16, 18. In one preferred embodiment, diameter 14 is about 6 mm. In another preferred embodiment, diameter 14 is about 5.5 mm. Diameter 14 is not limited to 5.5 mm or 6 mm but can be other values, depending upon the stiffness of the rod desired for a particular application, the hardware used for fixing the rod 10 to the spine, and the characteristics (e.g., size, condition, alignment/orientation, etc.) of the vertebrae to which the rod 10 is to be fixed. The length 20 of the rod 10/elongate body 12 will vary, depending upon how many levels of vertebrae the rod 10 is intended to attach to including any number of levels within the range of one to fourteen. Typically, length 20 will be in a range of from about 6 cm to about 60 cm, more typically in a range of from about 30 cm to 45 cm. In one example, length 20 is about 30 cm for spanning the T3-L2 vertebrae. In another example, length 20 is about 45 cm for spanning T2 through the sacrum. In another example, length 20 is about 60 cm for spanning T2 to the ilium.

FIG. 1B is a view of FIG. 1A in which an upper portion 22 of the elongate body 12 is shown as a longitudinal sectional view. This view illustrates the cavity or cannula 28 that is formed in the upper portion of the elongate body 12 to reduce the stiffness of portion 22, relative to the stiffness of the lower portion 24. The diameter 26 of the cavity 28 in the embodiment shown in FIG. 1B is about 3 mm, but can be in the range of about 1 mm to about 4 mm In the embodiment of FIG. 1B, the diameter 26 of cavity 28 is substantially constant over the length thereof. The length 30 of the cavity may vary, depending upon the amount of stiffness reduction is desired over the upper portion of the rod 10 and how many vertebrae it is desired to attach a less stiff portion of the rod to. In the embodiment of FIG. 1B, length 30 is about 75 mm, but may be in a range of from about 30 mm to about 100 mm Typically, one for levels (more typically, one to two levels) will be spanned by the less stiff portion of the rod at the top or at the bottom of a more stiff fusion construct. As shown, cavity 28 is concentric about the longitudinal axis 32 of rod 10 and rod 10 is also concentric about the longitudinal axis 32. FIG. 1C is a cross-sectional view of FIG. 1A taken along line 1C-1C and showing the cavity 28 surrounded by the elongate body 12. FIG. 1D is a cross-sectional view of FIG. 1A showing the solid lower portion of the elongate body 12. Alternatively, the rod 10 can be other than circular in cross-section, such as rectangular, oval, or other shape, or even a plate, but these conformations are less desirable as they would not be usable in deformity correction, and would be limited to a narrow range of spine fusion procedures. Further alternatively, rod 10 does not need to be concentric about the longitudinal axis, but could be offset. These alternatives are also less desirable for reasons provided above with regard to the shape of the cross-section.

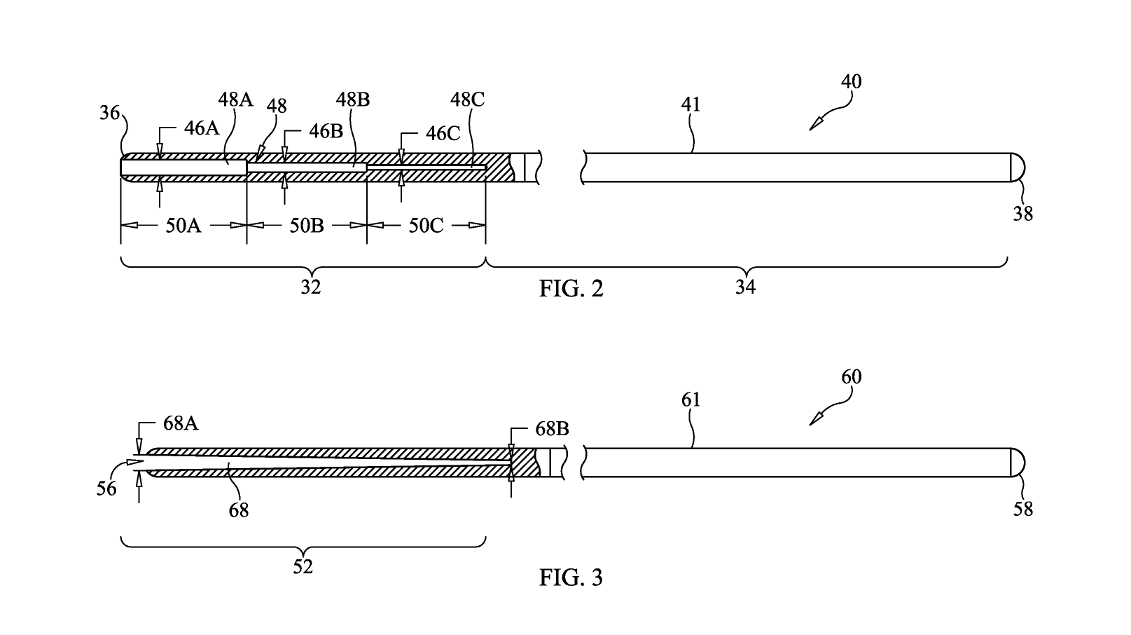

FIG. 2 is a view of a spinal rod 40 according to another embodiment of the present invention. Like FIG. 1B, FIG. 2 shows the upper portion of the elongate body 41 as a longitudinal sectional view. In this embodiment, the cavity 48 comprises stepped portions 48A, 48B, 48C. Although the embodiment of FIG. 2 shows three stepped portions, the cavity 48 could, alternatively have two stepped portions, or more than three stepped portions. Each stepped portion 48A, 48B, 48C has a diameter (46A, 46B, 46C, respectively) that is substantially constant over a length thereof. In the example shown in FIG. 2, diameter 48A is about 3.5 mm, diameter 48B is about 2.5 mm and diameter 48C is about 1.5 mm, although each of these dimensions may vary and be larger or smaller. In a preferred embodiment, as shown in FIG. 2, diameter 46A is larger than diameter 46B and diameter 46B is larger than diameter 46C. In a preferred embodiment, the stepped portion having the largest diameter is nearest the upper end 36 of the rod 40 and the diameters of the stepped portions are successively smaller from the largest diameter portion in a direction toward the lower end 38 of the rod 40. The length of each stepped portion can vary, and the percentage of the overall length of each portion, relative to the other portions can vary. In this way, the stiffness characteristics of portion 32 can be tailored as desired. The lengths 50A, 50B, 50C of the stepped portions in FIG. 2 are about 35 mm, 30 mm and 30 mm, respectively, although each may be varied to be longer or shorter. The length 32 of the portion of elongate body that contains the stepped portions 48A, 48B, 48C is about 95 mm in the embodiment of FIG. 2, and the overall length of the elongate body 41 is about 450 mm The length 32 relative to the overall length 32 plus 34 is typically about 21%, but may be in a range of from about 10% to about 50% of the overall length of the elongate body 41. A number of variant percentage lengths will be provided to accommodate different pathologies, bone quality, levels of fusion and patient heights. The lengths of portions 48A, 48B, 48C may all be equal, but are typically unequal, as each portion 48A, 48B, 48C, etc.(there may be more or less than three portions 48A, 48B, 48C) is typically designed to span one level.

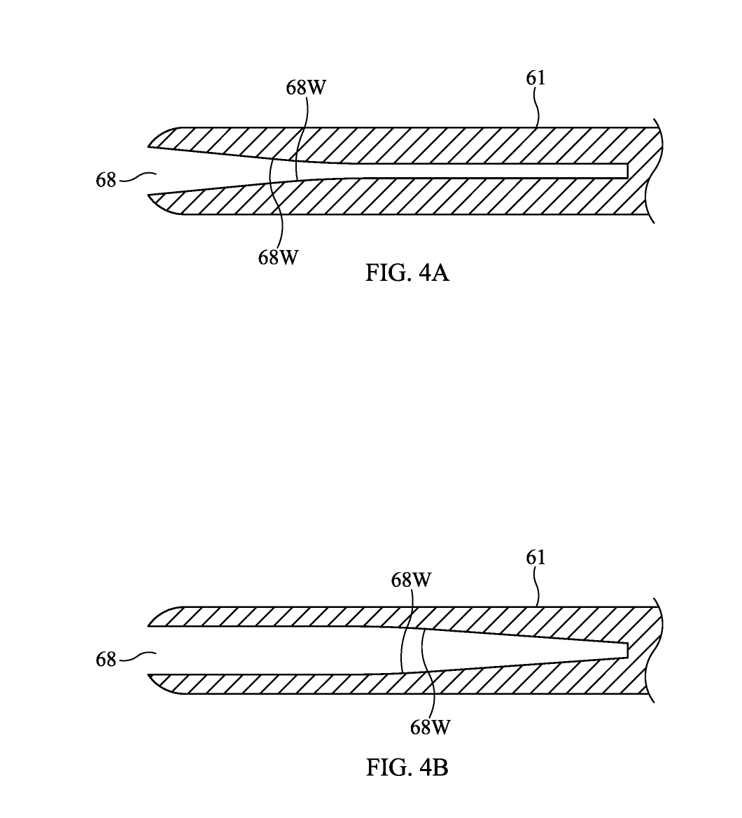

FIG. 3 is a view of a spinal rod 60 according to another embodiment of the present invention. Like FIG. 1B, FIG. 3 shows the upper portion of the elongate body 61 as a longitudinal sectional view. In this embodiment, the cavity 68 comprises a continuously tapering diameter that varies from a largest diameter 68A at the end of the cavity 68 nearest upper end 56 of the elongate body 61 to a smallest diameter 68B at the end of the cavity 68 nearest the lower end 58 of the elongate body. In the embodiment shown, diameter 68A is about 3.5 mm and diameter 68B is about 1 mm, although each of these diameters may be larger or smaller. The continuous taper of the diameter of the cavity 68 in the embodiment of FIG. 3 is linearly decreasing, as indicated by the straight lines of the cavity shown in the longitudinal sectional view of FIG. 3. Alternatively, the decreasing taper may be nonlinear, examples of which are illustrated in the partial, longitudinal, schematic views of FIG. 4A and 4B, showing convex curvature of the walls 68W of cavity 68 and concave curvature of walls 68W of cavity 68, respectively. Further alternatively, the walls 68W of cavity 68 can be formed in any shape, to further tailor the stiffness characteristics of portion 52 of the rod 60. There are an unlimited number of possibilities for design of the shapes and dimensions of the cavity walls 68W, as will be apparent to those of ordinary skill in the art after reading the present disclosure.

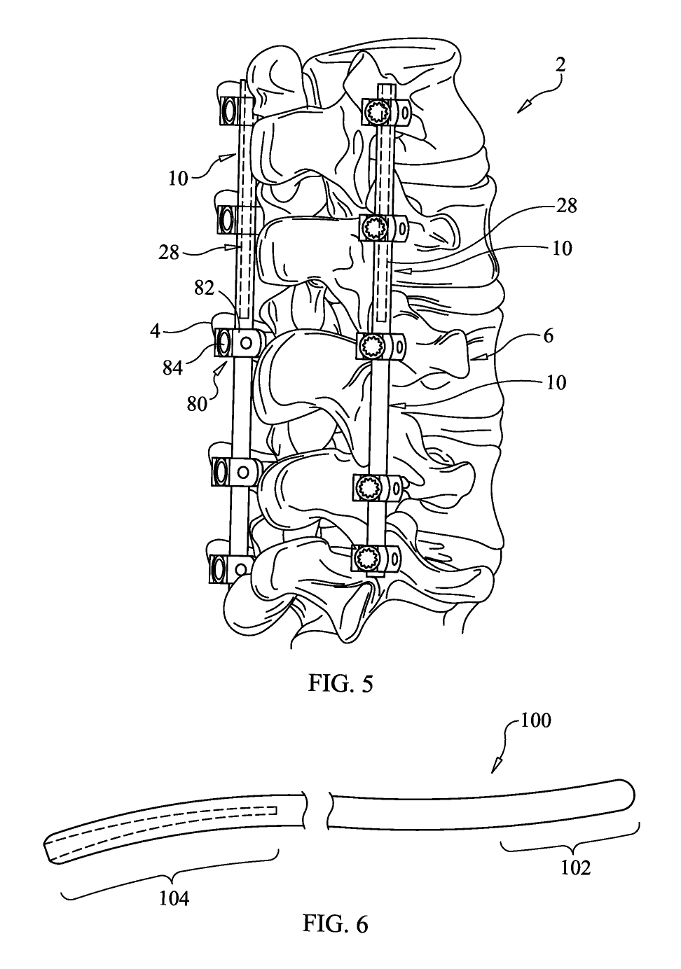

FIG. 5 illustrates a pair of spinal rods 10 having been implanted on the spine 2 of a patient according to an embodiment of the present invention. It is noted that spinal rods 40 and 60, and any embodiments of the present invention described herein, can be implanted in the same manner The spinal rods 10 are secured to the spine 2 by pedicle connectors 80 that include a pedicle screw 82 to secure the connector 80 to the pedicle 4 of a vertebra 6 and a retaining screw 84 that contacts the spinal rod 10 and compresses against it to lock it into position against the spine. Because the surface of the rod 10 is constant and does not taper in diameter, the retaining screw can establish full contact between its distal end surface and the surface of the rod 10 to establish a secure lock, along any location on the rod 10. This is not possible with a tapering rod, as the full surface of the distal end of the retaining screw cannot establish contact with a tapered surface. In the embodiment shown, a five level fusion is being performed, as the rods 10 are secured to five adjacent vertebrae, as shown. It is noted that the present invention is not limited to five-level fusions, as more or less vertebrae may be secured in a fusion, e.g., two-level, three-level, four level, or more than five-level, by selecting rods 10 of the appropriate length and securing them in the same manner as shown in FIG. 5. As noted, the present invention allows locking of the spinal rod to every vertebrae in the span of vertebrae being fused, using standard hardware, without the need to change sizes of pedicle connectors. It is further noted that the present invention is not limited to implanting a pair of spinal rods on the spine as shown in FIG. 5. Alternatively, a single rod could be implanted on one side of the spine in the same manner as described with regard to FIG. 5, with no second rod being implanted.

The embodiments of the present invention described this far have all been shown as straight rods. Alternatively, rods having the characteristics described above can be formed a curved rods, if desired. FIG. 6 illustrates a spinal rod that is curved along its length, according to an embodiment of the present invention. The lower portion 102 is curved in a first direction, and the upper portion is a curved in a second direction different from the first direction so that the rod 100 is configured to more closely conform to a curvature of the spine 2 that is desired to be achieved by the fusion. It is noted that the present invention is not limited to the curvatures shown I FIG. 6, as rod 100 could be made to have any curvature desired. Further alternatively, any of the spinal rods described herein can be made so that they can be bent by tools to deviate from a straight rod, so as to facilitate better alignment with a spinal curvature sought to be established by a fusion procedure.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the invention as described herein.

That which is claimed is:

1. A spinal rod comprising:
an elongate body having first and second ends, a length and an outside diameter, said outside diameter being substantially the same size over an entirety of said length of said elongate body; and
a cavity extending within said elongate body over a cavity length;
wherein said cavity length is less than said length of said elongate body; and
wherein a stiffness of said elongate body in portions containing said cavity is less than a stiffness of a portion of said elongate body not containing said cavity.

2. The spinal rod of claim 1, wherein said cavity length is in the range of 10% to 50% of said length of said elongate body.

3. The spinal rod of claim 2, wherein said cavity length is in the range about 15% to about 35% of said length of said elongate body.

4. The spinal rod of claim 1, wherein said cavity length is less than 50% of said length of said elongate body.

5. The spinal rod of claim 1, wherein said cavity extends from said first end to a location intermediate of said first and second ends.

6. The spinal rod of claim 1, wherein a diameter of said cavity varies along a length of said cavity.

7. The spinal rod of claim 1, wherein said cavity includes stepped portions, each said stepped portion having a diameter that is substantially constant over a length of said respective step portion, and wherein said diameters of said stepped portions are each different from one another.

8. The spinal rod of claim 7, wherein said diameter of a first of said stepped portions is larger than said diameters of all other of said stepped portions and extends from said first end of said main body.

9. The spinal rod of claim 8, wherein said diameters of said stepped portions become decreasingly smaller in a direction from said first of said stepped portions toward said second end of said elongate body.

10. The spinal rod of claim 6, wherein said diameter of said cavity varies continuously over a length thereof.

11. The spinal rod of claim 10, wherein said diameter of said cavity decreases from a location nearest said first end of said elongate body to a location nearest said second end of said elongate body.

12. The spinal rod of claim 1, wherein said spinal rod is made entirely of a single material.

13. The spinal rod of claim 12, wherein said single material is metal.

14. The spinal rod of claim 13, wherein said metal comprises titanium.

15. The spinal rod of claim 13, wherein said metal comprises Cobalt Chromium.

16. The spinal rod of claim 12, wherein said single material comprises polymer.

17. The spinal rod of claim 1, wherein a first portion of said elongate body contains said cavity and a second portion of said elongate body is solid, and wherein said first portion has a stiffness in a range of from about 10 N/mm to about 250 N/mm and said second portion has a stiffness in a range of from about 40 N/mm to about 1000 N/mm.

18. The spinal rod of claim 1,
wherein a first portion of said elongate body contains said cavity and a second portion of said elongate body is solid;
wherein said second portion has a stiffness in a range of from about 40 N/mm to about 1000 N/mm;
wherein said first portion has at least first and second sub-portions, such that said first sub-portion portion has a stiffness in a range of from about 20 N/mm to about 500 N/mm and said second sub-portion has a stiffness in a range of from about 10 N/mm to about 250 N/mm; and
wherein said stiffness of said first sub-portion is greater than said stiffness of said second sub-portion.

19. The spinal rod of claim 18, wherein said first-sub portion is located intermediate of said second portion and said second sub-portion.

20. The spinal rod of claim 1, further comprising a marker on an external surface of said elongate body, said marker identifying a location intermediate said first and second ends where said cavity ends.

21. A method of performing spinal fusion, said method comprising:
selecting a spinal rod comprising an elongate body having first and second ends, a length and an outside diameter, the outside diameter being substantially the same size over an entirety of the length of the elongate body; and a cavity extending within the elongate body over a cavity length, wherein the cavity length is less than the length of the elongate body; and
fixing the spinal rod to at least two adjacent spinal vertebrae.

22. The method of claim 21, wherein said fixing comprises fixing the spinal rod to three or more adjacent vertebrae.

23. The method of claim 21, wherein said fixing comprises orienting the spinal rod so that a portion of the elongate body containing the cavity is above a portion of the elongate body that does not contain the cavity.

24. The method of claim 21, wherein said fixing comprises fixing the spinal rod to each of the vertebrae spanned by the elongate body.
